(19) United States
(12) Patent Application Publication
(10) Pub. No.: US 2013/0078212 A1
(43) Pub. Date: Mar. 28, 2013

(54) Double Stranded RNA Constructs to Control Ants

(71) Applicants: Liming Zhao, Gainesville, FL (US); Jian Chen, Greenville, MS (US)

(72) Inventors: Liming Zhao, Gainesville, FL (US); Jian Chen, Greenville, MS (US)

(21) Appl. No.: 13/609,582

(22) Filed: Sep. 11, 2012

Related U.S. Application Data
(60) Provisional application No. 61/540,034, filed on Sep. 28, 2011.

(51) Int. Cl. C07H 21/02; A01P 7/04; A01N 43/04

(52) U.S. Cl. 424/84; 536/24.5; 514/44 A

(57) ABSTRACT

Disclosed are double stranded RNA constructs used to inhibit the expression of guanine nucleotide binding (3-subunit to induce mortality in ants classified in the Formicidae family.

### CROSS-REFERENCE TO RELATED APPLICATION

This present application claims priority under 35 U.S.C. §119(e) to U.S. Provisional Serial No. 61/540,034, which was filed on Sep. 28, 2011, and is hereby incorporated by reference.

### FIELD OF THE INVENTION

This invention relates to double stranded RNA constructs to inhibit the expression of guanine nucleotide binding β-subunit to induce mortality in ants classified in the Formicidae family.

### BACKGROUND OF INVENTION

Insect pests cost the general public billions of dollars annually in losses. These losses include the expense of controlling insect pests as well as crop loss and property damage caused by the pests. Specifically ants comprise 5% of the world's hundred worst invasive alien species as reported in Lo S., Browne M., Boudjelas S., De Poorter M, (2000) 100 of the World's Worst Invasive Alien Species A selection from the Global Invasive Species Database. Published by The Invasive Species Specialist Group (ISSG) a specialist group of the Species Survival Commission (SSC) of the World Conservation Union (IUCN), 12 pp. First published as special lift-out in Aliens 12, December 2000 and electronically available at http://www.issg.org/database/species/search.asp?st=100ss. Of the 17 land invertebrates listed, 28% are ants, including fire ants (Solenopsis spp.), Argentine ant (Linepithema humile), the little fire ant (Wasmannia auropunctata), and the crazy ant (Paratrechina spp). All of these ants have substantial economic impact. More specifically, the well-documented fire ant currently infests over 320 million acres in the United States and over $6 billion per year is spent for control and damage repair (as reported in Lard, C. F., J. Schmidt, B. Morris, L. Estes, C. Ryan, and D. Bergquist. 2006. “An economic impact of imported fire ants in the United States of America.” Texas A&M University, College Station, Texas. Available online at http://fireantecon.tamu.edu). The economic sectors affected include: residential households, electric and communication systems, agriculture (crops and livestock), golf courses, commercial businesses, schools and medical facilities, and parks and recreational areas.

The red imported fire ant, Solenopsis invicta Buren (Hymenoptera: Formicidae), was introduced from Brazil into United States in the 1930's and have been found in many southern and western parts of the United States from Maryland to southern California. The red imported fire ant has become a major agricultural and urban pest throughout those parts of the United States as S. invicta can cause significant damage to soybean, citrus, corn, okra, bean, cabbage, cucumber, eggplant, potato, sweet potato, peanut, sorghum, cotton and sunflower. Their mound-building activity can damage plant roots, leading to crop loss as well as interference with mechanical cultivation of crops.

Chemical pesticides are the primary tools used to combat S. invicta. However the use of traditional chemical pesticides has disadvantages, including non-target effects on neutral or beneficial insects, as well as other animals. Chemical pesticide usage also can lead to chemical residue run-off into streams and seepage into water supplies resulting in ecosystem/environment damage. In addition, animals higher in the food chain are at risk when they consume pesticide contaminated crops or insects. The handling and application of chemical pesticides also presents exposure danger to the public and professionals, and could lead to accidental dispersal into unintended environmentally sensitive areas. In addition, prolonged chemical pesticide application may result in an insect population becoming resistance to a chemical pesticide. In order to control a traditionally chemical resistant-pest, new more potent chemical pesticides must be utilized, which in turn will lead to another resistance cycle. As such, there is a need in the art to control pest populations without the disadvantages of traditional chemical pesticides.

An approach to decrease dependence on chemical pesticides is by causing a specific gene(s) of the target-pest to malfunction by either over expression or silencing gene expression. The silencing approach utilizes RNA interference pathways to knockdown the gene of interest via double strand RNA. Double strand RNA (dsRNA) induces sequence—specific post-transcriptional gene silencing in many organisms by a process known as RNA interference (RNAi). RNAi is a post-transcriptional, highly conserved process in eukaryotes that leads to specific gene silencing through degradation of the target mRNA. The silencing mechanism is mediated by dsRNA that is homologous in sequence to the gene of interest. The dsRNA is processed into small interfering RNA (siRNA) by an endogenous enzyme called DICER inside the target pest, and the siRNAs are then incorporated into a multi-component RNA-induced silencing complex (RISC), which finds and cleaves the target mRNA. The dsRNA inhibits expression of at least one gene within the target, which exerts a deleterious effect upon the target.

Fire, et al. (U.S. Pat. No. 6,506,559) discloses a process of introducing RNA into a living cell to inhibit gene expression of a target gene in that cell. The RNA has a region with double-stranded structure. Inhibition is sequence-specific in that the nucleotide sequences of the duplex region of the RNA and of a portion of the target gene are identical. Specifically, Fire, et al. (U.S. Pat. No. 6,506,559) discloses a method to inhibit expression of a target gene in a cell, the method comprising introduction of a double stranded ribonucleic acid into the cell in an amount sufficient to inhibit expression of the target gene, wherein the RNA is a double-stranded molecule with a first ribonucleic acid strand consisting essentially of a ribonucleotide sequence which corresponds to a nucleotide sequence of the target gene and a second ribonucleic acid strand consisting essentially of a ribonucleotide sequence which is complementary to the nucleotide sequence of the target gene. Furthermore, the first and the second ribonucleotide strands are separately complementary strands that hybridize to each other to form the said double-stranded construct, and the double-stranded construct inhibits expression of the target gene.

In using dsRNA in controlling a target insect, one method is to engineer a baculovirus to produce a dsRNA construct in vivo as disclosed in Liu, et al. (U.S. Pat. No. 6,846,482). Salient to Liu is contacting an insect with a recombinant baculovirus wherein a first ribonucleic acid sequence corresponds to at least a portion of at least one gene endogenous to the insect to control the insect. Given the advances made in the field of transfection efficiency and RNA interference, there is a need in the art to utilize RNA interference technology without using a baculovirus as a vector. Such a method would mediate control of a target-pest without depending on variables associated with a baculovirus, such as expression and transfection of dsRNA by the baculovirus.

To utilize RNA interference as a method to regulate gene expression to control a target organism, a specific essential gene needs to be targeted. Genes associated with guanine nucleotide binding protein (GNBP) represent a novel potential target for Solenopsis invicta. GNBPs are glycoproteins anchored on the cytoplasmic cell membrane and mediate cellular processes such as signal transduction in cells. Guanine nucleotide binding proteins (GNBP or G-protein), known as GTP-binding proteins and GTPases, are glycoproteins anchored on the cytoplasmic cell membrane, and are mediators for many cellular processes, including signal transduction, protein transport, growth regulation, and polypeptide chain elongation. Gbeta subunits from heterotrimeric G-proteins directly bind diverse proteins, including effectors and regulators, to modulate a wide array of signaling cascades. G-proteins have been identified in a variety of animals, plants, fungi, and insects, including Caenorhabditis elegans, Drosophila melanogaster, Bombyx mori, Spodoptera exigua.

Given the broad application of GNBP, there is a need in the art to investigative whether the interference of the silencing of GNBP mRNA can be used to control insects within the Formicidae family, such Solenopsis invicta.

Such novel control methods that would induce silencing of GNBP would be desirable as the undesirable characteristics of traditional chemical pesticides. Traditional chemical pesticides in general have the disadvantage of being toxic to the environment as well as affecting a broad range of insect. To that end, there is a need to develop dsRNA constructs that are engineered to silence target GNBP mRNA that would overcome some of the disadvantages of using traditional pesticides and that can target specific target pests.

### BRIEF SUMMARY OF THE INVENTION

Disclosed herein is a double-stranded ribonucleic acid (dsRNA) for inhibiting the expression of a guanine nucleotide binding protein in a cell, wherein said dsRNA comprises SEQ ID NO: 29 and an antisense strand comprising a sequence complementary to SEQ ID NO: 29. In one embodiment, the antisense strand is SEQ ID NO 30.

Also disclosed herein is a double-stranded ribonucleic acid (dsRNA) for inhibiting the expression of a guanine nucleotide binding protein in a cell, wherein said dsRNA comprises SEQ ID NO: 31 and an antisense strand comprising a sequence complementary to SEQ ID NO: 31. In one embodiment, the antisense strand is SEQ ID NO 32.

Also disclosed herein is a double-stranded ribonucleic acid (dsRNA) for inhibiting the expression of a guanine nucleotide binding protein in a cell, wherein said dsRNA comprises

SEQ ID NO: 33 and an antisense strand comprising a sequence complementary to SEQ ID NO: 33. In one embodiment, the antisense strand is SEQ ID NO 34.

Also disclosed herein is a double-stranded ribonucleic acid (dsRNA) for inhibiting the expression of a guanine nucleotide binding protein in a cell, wherein said dsRNA comprises SEQ ID NO: 35 and an antisense strand comprising a sequence complementary to SEQ ID NO: 35. In one embodiment, the antisense strand is SEQ ID NO 36.

Also disclosed herein is a double-stranded ribonucleic acid (dsRNA) for inhibiting the expression of a guanine nucleotide binding protein in a cell, wherein said dsRNA comprises SEQ ID NO: 37 and an antisense strand comprising a sequence complementary to SEQ ID NO: 37. In one embodiment, the antisense strand is SEQ ID NO 38.

Also disclosed herein is a double-stranded ribonucleic acid (dsRNA) for inhibiting the expression of a guanine nucleotide binding protein in a cell, wherein said dsRNA comprises SEQ ID NO: 39 and an antisense strand comprising a sequence complementary to SEQ ID NO: 39. In one embodiment, the antisense strand is SEQ ID NO 40.

Disclosed herewith is a method for controlling Solenopsis invicta, the method comprising: constructing a double stranded ribonucleic acid construct that is complementary to a gene that encodes a guanine nucleotide binding protein, dissolving the double stranded ribonucleic acid to form a solution, and contacting an effective amount of said solution to Solenopsis invicta, wherein said solution is ingested by Solenopsis invicta and RNA interference is induced, resulting in mortality of Solenopsis invicta.

In one embodiment of the invention, one strand of the double stranded ribonucleic acid is used to control Solenopsi invicta, wherein the double stranded ribonucleic acid is complementary to the nucleotide sequence selected from the group consisting of SEQ ID NO: 29, SEQ ID NO: 31, SEQ ID NO: 33, SEQ ID NO: 35, SEQ ID NO: 37, and SEQ ID NO: 39.

In an embodiment of the invention, the double stranded ribonucleic acid is complementary to the nucleotide sequence of SEQ ID NO: 29, SEQ ID NO: 31, SEQ ID NO: 33, SEQ ID NO: 35, SEQ ID NO: 37, and SEQ ID NO: 39. In another embodiment of the invention, the double stranded ribonucleic acid construct is dissolved in a sucrose solution. In yet another embodiment of the invention, the double stranded ribonucleic acid construct is dissolved in water.

In an embodiment of the invention, the double stranded ribonucleic acid construct is applied to Solenopsis invicta bait material. In various embodiments of the bait material, the bait material is a granular bait. In different embodiments of the bait material, the bait material can be a solution or granules that attract a target insect.

In another embodiment of the invention, a double stranded ribonucleic acid construct is mixed with a solution, wherein the solution is applied topically to control Solenopsis invicta. In one embodiment of the invention, the solution containing the double stranded ribonucleic acid construct is fed to Solenopsis invicta workers.

In another embodiment of the invention, a double stranded ribonucleic acid construct is mixed with a solution, wherein the solution is fed to Solenopsis invicta larvae.

### BRIEF DESCRIPTION OF THE DRAWING

The present invention together with the disclosed embodiments may best be understood from the following detailed description of the drawings, wherein:

FIG. 1 is a graph depicting qPCR results showing the relative ratio of SiGNBP mRNA expressed (down-regulated) in the worker S. invicta at 12 h after liquid feeding assay initiated with in vitro synthesized dsRNA-SiGNBPA-F products compared with the control with SD (standard deviation) for three replicates. Cont=control, A=dsRNA-SiGNBP-A, B=dsRNA-SiGNBP-B, C=dsRNA-SiGNBP-C, D=dsRNA-SiGNBP-D, E=dsRNA-SiGNBP-E, and F=dsRNA-SiGNBP-F.

FIG. 2 is a graph depicting qPCR results showing the relative ratio of SiGNBP mRNA expressed (down-regulated) in the worker S. invicta at 12 h after granular feeding assay initiated with in vitro synthesized dsRNA-SiGNBPA-F products compared with the control with SD for three replicates. Cont=control, A=dsRNA-SiGNBP-A, B=dsRNA-SiGNBP-B, C=dsRNA-SiGNBP-C, D=dsRNA-SiGNBP-D, E=dsRNA-SiGNBP-E, and F=dsRNA-SiGNBP-F.

### BRIEF DESCRIPTION OF THE SEQUENCES

SEQ ID NO: 1 is a 5′ to 3′ construct from primers T7-SiGNBP-13F / T7-SiGNBP-267R cDNA template used to form one strand of the dsRNA product referred to as dsRNA-SiGNBP-A:

TTACAGCTGAGAGGGACGCTTCGCGGCCACAATGGATGGGTCACGCAA
ATCGCGACAAACCCGAAATATCCAGACATGATTTTGTCTTCTTCACGT
GATAAGACTCTGATTGTGTGGAAATTGACTCGTGATGAAGCTAACTAT
GGTATCCCGCAGAAGCGTCTCTATGGTCACTCACACTTCATAAGTGAT
GTAGTTCTTTCATCTGATGGTAACTACGCTCTGTCTGGTTCATGGGAC
AAAACATTGCGTCTT.

SEQ ID NO: 2 is a 5′ to 3′ construct from primers T7-SiGNBP-248F / T7-SiGNBP-361R cDNA template used to form one strand of the dsRNA product referred to as dsRNA-SiGNBP-B:

GGGACAAAACATTGCGTCTTTGGGATCTCGCAGCAGGTCGTACGACCA
GACGATTTGAAGACCATACCAAGGATGTTTTGAGTGTCGCTTTTTCCG
TGGACAATCGTCAGATTG.

SEQ ID NO: 3 is a 5′ to 3′ construct from primers T7-SiGNBP-361F / T7-SiGNBP-653R cDNA template used to form one strand of the dsRNA product referred to as dsRNA-SiGNBP-C:

CGTGGACAATCGTCAGATTGTTTCCGGTTCGCGAGACAAGACAATTAA
ATTGTGGAATACATTGGCCGAATGCAAGTATACCATCCAGGATGATGG
GCATACAGATTGGGTCAGCTGTGTGCGCTTCTCCCCCAATCATGCAAA
TCCCATCATTGTCTCTGCAGGCTGGGATCGCGTTGTCAAGGTCTGGAA
CTTAACGAACTGCAGGTTGAAGATCAATCATAGTGGTCATACAGGATA
TCTGAATACAGTTACGGTATCGCCTGATGGATCACTCTGCGCATCCGG
TGGAAAAGATTGCAAAGCTATGCTGTGGGATCTGAATGATGGA.

SEQ ID NO: 4 is a 5′ to 3′ construct from primers T7-SiGNBP-653F / T7-SiGNBP-940R cDNA template used to form forming one strand of the dsRNA product referred to as dsRNA-SiGNBP-D:

TGTGGGATCTGAATGATGGAAAACATCTCCACACTTTAGACCATAATG
ACATCATTACGGCATTATGCTTTAGTCCTAATCGTTATTGGCTGTGCG
CCGCATTCGGACCATGGATTAAGATATGGGATCTTGAGACTAAGGAAA
TGGTTGAAGAACTGAAACCTGAGGTTGTGTCTGCAACTAGTAAAGCAG
AGCCACCTCTCTGTTTGTCCCTCGCGTGGTCCACCGACGGTCAAACGT
TGTTCGCTGGATACTCCGATAATACTATTCGTGTCTGGCAAGTTTCTG.

SEQ ID NO: 5 is a 5′ to 3′ construct from primers T7-SiGNBP-1F / T7-SiGNBP-361R cDNA template used to form one strand of the dsRNA product referred to as dsRNA-SiGNBP-E:

ATGACCGAGACTTTACAGCTGAGAGGGACGCTTCGCGGCCACAATGGA
TGGGTCACGCAAATCGCGACAAACCCGAAATATCCAGACATGATTTTG
TCTTCTTCACGTGATAAGACTCTGATTGTGTGGAAATTGACTCGTGAT
GAAGCTAACTATGGTATCCCGCAGAAGCGTCTCTATGGTCACTCACAC
TTCATAAGTGATGTAGTTCTTTCATCTGATGGTAACTACGCTCTGTCT
GGTTCATGGGACAAAACATTGCGTCTTTGGGATCTCGCAGCAGGTCGT
ACGACCAGACGATTTGAAGACCATACCAAGGATGTTTTGAGTGTCGCT
TTTTCCGTGGACAATCGTCAGATTG.

SEQ ID NO: 6 is a 5′ to 3′ construct from primers T7-SiGNBP-361F / T7-SiGNBP-940R cDNA template used to form one strand of the dsRNA product referred to as dsRNA-SiGNBP-F:

CGTGGACAATCGTCAGATTGTTTCCGGTTCGCGAGACAAGACAATTAA
ATTGTGGAATACATTGGCCGAATGCAAGTATACCATCCAGGATGATGG
GCATACAGATTGGGTCAGCTGTGTGCGCTTCTCCCCCAATCATGCAAA
TCCCATCATTGTCTCTGCAGGCTGGGATCGCGTTGTCAAGGTCTGGAA
CTTAACGAACTGCAGGTTGAAGATCAATCATAGTGGTCATACAGGATA
TCTGAATACAGTTACGGTATCGCCTGATGGATCACTCTGCGCATCCGG
TGGAAAAGATTGCAAAGCTATGCTGTGGGATCTGAATGATGGAAAACA
TCTCCACACTTTAGACCATAATGACATCATTACGGCATTATGCTTTAG
TCCTAATCGTTATTGGCTGTGCGCCGCATTCGGACCATGGATTAAGAT
ATGGGATCTTGAGACTAAGGAAATGGTTGAAGAACTGAAACCTGAGGT
TGTGTCTGCAACTAGTAAAGCAGAGCCACCTCTCTGTTTGTCCCTCGC
GTGGTCCACCGACGGTCAAACGTTGTTCGCTGGATACTCCGATAATAC
TATTCGTGTCTGGCAAGTTTCTG.

SEQ ID NO: 7

ATGACCGAGACTTTACAGCTGAGAGGGACGCTTCGCGGCCACAATGGA
TGGGTCACGCAAATCGCGACAAACCCGAAATATCCAGACATGATTTTG
TCTTCTTCACGTGATAAGACTCTGATTGTGTGGAAATTGACTCGTGAT
GAAGCTAACTATGGTATCCCGCAGAAGCGTCTCTATGGTCACTCACAC
TTCATAAGTGATGTAGTTCTTTCATCTGATGGTAACTACGCTCTGTCT
GGTTCATGGGACAAAACATTGCGTCTTTGGGATCTCGCAGCAGGTCGT
ACGACCAGACGATTTGAAGACCATACCAAGGATGTTTTGAGTGTCGCT
TTTTCCGTGGACAATCGTCAGATTGTTTCCGGTTCGCGAGACAAGACA
ATTAAATTGTGGAATACATTGGCCGAATGCAAGTATACCATCCAGGAT
GATGGGCATACAGATTGGGTCAGCTGTGTGCGCTTCTCCCCCAATCAT
GCAAATCCCATCATTGTCTCTGCAGGCTGGGATCGCGTTGTCAAGGTC
TGGAACTTAACGAACTGCAGGTTGAAGATCAATCATAGTGGTCATACA
GGATATCTGAATACAGTTACGGTATCGCCTGATGGATCACTCTGCGCA
TCCGGTGGAAAAGATTGCAAAGCTATGCTGTGGGATCTGAATGATGGA
AAACATCTCCACACTTTAGACCATAATGACATCATTACGGCATTATGC
TTTAGTCCTAATCGTTATTGGCTGTGCGCCGCATTCGGACCATGGATT
AAGATATGGGATCTTGAGACTAAGGAAATGGTTGAAGAACTGAAACCT
GAGGTTGTGTCTGCAACTAGTAAAGCAGAGCCACCTCTCTGTTTGTCC
CTCGCGTGGTCCACCGACGGTCAAACGTTGTTCGCTGGATACTCCGAT
AATACTATTCGTGTCTGGCAAGTTTCTGTATCCAGCAGATAA

is the cDNA sequence of Solenopsis invicta deposited in Genbank (Accession Number HM130685).

SEQ ID NO: 8 is a 5′ to 3′ construct from primers T7-SiGNBP-13F / T7-SiGNBP-267R cDNA template used to form one strand of the dsRNA product referred to as dsRNA-SiGNBP-A, with SEQ ID NO: 8 being complementary to SEQ ID NO: 1:

AAGACGCAATGTTTTGTCCCATGAACCAGACAGAGCGTAGTTACCATC
AGATGAAAGAACTACATCACTTATGAAGTGTGAGTGACCATAGAGACG
CTTCTGCGGGATACCATAGTTAGCTTCATCACGAGTCAATTTCCACAC
AATCAGAGTCTTATCACGTGAAGAAGACAAAATCATGTCTGGATATTT
CGGGTTTGTCGCGATTTGCGTGACCCATCCATTGTGGCCGCGAAGCGT
CCCTCTCAGCTGTAA.

SEQ ID NO: 9 is a 5′ to 3′ construct from primers T7-SiGNBP-248F / T7-SiGNBP-361R cDNA template used to form one strand of the dsRNA product referred to as dsRNA-SiGNBP-B, with SEQ ID NO: 9 being complementary to SEQ ID NO: 2:

CAATCTGACGATTGTCCACGGAAAAAGCGACACTCAAAACATCCTTGG
TATGGTCTTCAAATCGTCTGGTCGTACGACCTGCTGCGAGATCCCAAA
GACGCAATGTTTTGTCCC.

SEQ ID NO: 10 is a 5′ to 3′ construct from primers T7-SiGNBP-361F / T7-SiGNBP-653R cDNA template used to form one strand of the dsRNA product referred to as dsRNA-SiGNBP-C, with SEQ ID NO: 10 being complementary to SEQ ID NO: 3:

TCCATCATTCAGATCCCACAGCATAGCTTTGCAATCTTTTCCACCGGA
TGCGCAGAGTGATCCATCAGGCGATACCGTAACTGTATTCAGATATCC
TGTATGACCACTATGATTGATCTTCAACCTGCAGTTCGTTAAGTTCCA
GACCTTGACAACGCGATCCCAGCCTGCAGAGACAATGATGGGATTTGC
ATGATTGGGGGAGAAGCGCACACAGCTGACCCAATCTGTATGCCCATC
ATCCTGGATGGTATACTTGCATTCGGCCAATGTATTCCACAATTTAAT
TGTCTTGTCTCGCGAACCGGAAACAATCTGACGATTGTCCACG.

SEQ ID NO: 11 is a 5′ to 3′ construct from primers T7-SiGNBP-653F / T7-SiGNBP-940R cDNA template used to form one strand of the dsRNA product referred to as dsRNA-SiGNBP-D, with SEQ ID NO: 11 being complementary to SEQ ID NO: 4:

CAGAAACTTGCCAGACACGAATAGTATTATCGGAGTATCCAGCGAACA
ACGTTTGACCGTCGGTGGACCACGCGAGGGACAAACAGAGAGGTGGCT
CTGCTTTACTAGTTGCAGACACAACCTCAGGTTTCAGTTCTTCAACCA
TTTCCTTAGTCTCAAGATCCCATATCTTAATCCATGGTCCGAATGCGG
CGCACAGCCAATAACGATTAGGACTAAAGCATAATGCCGTAATGATGT
CATTATGGTCTAAAGTGTGGAGATGTTTTCCATCATTCAGATCCCACA.

SEQ ID NO: 12 is a 5′ to 3′ construct from primers T7-SiGNBP-1F / T7-SiGNBP-361R cDNA template used to form one strand of the dsRNA product referred to as dsRNA-SiGNBP-E, with SEQ ID NO: 12 being complementary to SEQ ID NO: 5:

CAATCTGACGATTGTCCACGGAAAAAGCGACACTCAAAACATCCTTGG
TATGGTCTTCAAATCGTCTGGTCGTACGACCTGCTGCGAGATCCCAAA
GACGCAATGTTTTGTCCCATGAACCAGACAGAGCGTAGTTACCATCAG
ATGAAAGAACTACATCACTTATGAAGTGTGAGTGACCATAGAGACGCT
TCTGCGGGATACCATAGTTAGCTTCATCACGAGTCAATTTCCACACAA
TCAGAGTCTTATCACGTGAAGAAGACAAAATCATGTCTGGATATTTCG
GGTTTGTCGCGATTTGCGTGACCCATCCATTGTGGCCGCGAAGCGTCC
CTCTCAGCTGTAAAGTCTCGGTCAT.

SEQ ID NO: 13 is a 5′ to 3′ construct from primers T7-SiGNBP-361F / T7-SiGNBP-940R cDNA template used to form one strand of the dsRNA product referred to as dsRNA-SiGNBP-F, with SEQ ID NO: 13 being complementary to SEQ ID NO: 6:

CAGAAACTTGCCAGACACGAATAGTATTATCGGAGTATCCAGCGAACA
ACGTTTGACCGTCGGTGGACCACGCGAGGGACAAACAGAGAGGTGGCT
CTGCTTTACTAGTTGCAGACACAACCTCAGGTTTCAGTTCTTCAACCA
TTTCCTTAGTCTCAAGATCCCATATCTTAATCCATGGTCCGAATGCGG
CGCACAGCCAATAACGATTAGGACTAAAGCATAATGCCGTAATGATGT
CATTATGGTCTAAAGTGTGGAGATGTTTTCCATCATTCAGATCCCACA
GCATAGCTTTGCAATCTTTTCCACCGGATGCGCAGAGTGATCCATCAG
GCGATACCGTAACTGTATTCAGATATCCTGTATGACCACTATGATTGA
TCTTCAACCTGCAGTTCGTTAAGTTCCAGACCTTGACAACGCGATCCC
AGCCTGCAGAGACAATGATGGGATTTGCATGATTGGGGGAGAAGCGCA
CACAGCTGACCCAATCTGTATGCCCATCATCCTGGATGGTATACTTGC
ATTCGGCCAATGTATTCCACAATTTAATTGTCTTGTCTCGCGAACCGG
AAACAATCTGACGATTGTCCACG.

SEQ ID NO: 14 is primer T7-SiGNBP-1F used to synthesis dsRNA products, SEQ ID

NO: 14: TAATACGACTCACTATAGGGATGACCGAGACTTTACAGCT.

SEQ ID NO: 15 is primer T7-SiGNBP-267R used to synthesis dsRNA products, SEQ ID NO: 15: TAATACGACTCACTATAGGGAAGACGCAATGTTTTGTCCC.

SEQ ID NO: 16 is primer T7-SiGNBP-653F used to synthesis dsRNA products, SEQ ID NO: 16: TAATACGACTCACTATAGGGTGTGGGATCTGAATGATGGA.

SEQ ID NO: 17 is primer T7-SiGNBP-940R used to synthesis dsRNA products, SEQ ID NO: 17: TAATACGACTCACTATAGGGCAGAAACTTGCCAGACACGA.

SEQ ID NO: 18 is primer T7-SiGNBP-13F used to synthesis dsRNA products, SEQ ID NO: 18: TAATACGACTCACTATAGGGTTACAGCTGAGAGGGACGCT.

SEQ ID NO: 19 is primer T7-SiGNBP-248F used to synthesis dsRNA products, SEQ ID NO: 19: TAATACGACTCACTATAGGGGGGACAAAACATTGCGTCTT

SEQ ID NO: 20 is primer T7-SiGNBP-361R used to synthesis dsRNA products, SEQ ID NO: 20: TAATACGACTCACTATAGGGCAATCTGACGATTGTCCACG.

SEQ ID NO: 21 is primer T7-SiGNBP-361F used to synthesis dsRNA products, SEQ ID NO: 21: TAATACGACTCACTATAGGGCGTGGACAATCGTCAGATTG.

SEQ ID NO: 22 is primer T7-SiGNBP-653R used to synthesis dsRNA products, SEQ ID NO: 22: TAATACGACTCACTATAGGGTCCATCATTCAGATCCCACA.

SEQ ID NO: 23 is primer SiActin-783-F used for qPCR, SEQ ID NO: 23: CCTCTTCCAACCTTCCTTCC.

SEQ ID NO: 24 is primer SiActin-948R used for qPCR, SEQ ID NO: 24: CTTTTGCATACGATCAGCGA.

SEQ ID NO: 25 is primer SiActin-605F used for qPCR, SEQ ID NO: 25: GGTCGCGATCTTACCGATTA.

SEQ ID NO: 26 is primer SiActin-859R used for qPCR, SEQ ID NO: 26: GAAGGAAGGTTGGAAGAGGG.

SEQ ID NO: 27 is primer SiGNBP-13F used for qPCR, SEQ ID NO: 27: TTACAGCTGAGAGGGACGCT.

SEQ ID NO: 28 is primer SiGNBP-267R used for qPCR, SEQ ID NO: 28: AAGACGCAATGTTTTGTCCC.

SEQ ID NO: 29 is a 5′ to 3′ RNA construct from primers T7-SiGNBP-13F / T7-SiGNBP-267R forming one strand of the dsRNA product referred to as dsRNA-SiGNBP-A:

UUACAGCUGAGAGGGACGCUUCGCGGCCACAAUGGAUGGGUCACGCAA
AUCGCGACAAACCCGAAAUAUCCAGACAUGAUUUUGUCUUCUUCACGU
GAUAAGACUCUGAUUGUGUGGAAAUUGACUCGUGAUGAAGCUAACUAU
GGUAUCCCGCAGAAGCGUCUCUAUGGUCACUCACACUUCAUAAGUGAU
GUAGUUCUUUCAUCUGAUGGUAACUACGCUCUGUCUGGUUCAUGGGAC
AAAACAUUGCGUCUU.

SEQ ID NO: 30 is a 5′ to 3′ RNA construct from primers T7-SiGNBP-13F / T7-SiGNBP-267R forming one strand of the dsRNA product referred to as dsRNA-SiGNBP-A, with SEQ ID NO: 30 being complementary to SEQ ID NO: 29:

AAGACGCAAUGUUUUGUCCCAUGAACCAGACAGAGCGUAGUUACCAUC
AGAUGAAAGAACUACAUCACUUAUGAAGUGUGAGUGACCAUAGAGACG
CUUCUGCGGGAUACCAUAGUUAGCUUCAUCACGAGUCAAUUUCCACAC
AAUCAGAGUCUUAUCACGUGAAGAAGACAAAAUCAUGUCUGGAUAUUU
CGGGUUUGUCGCGAUUUGCGUGACCCAUCCAUUGUGGCCGCGAAGCGU
CCCUCUCAGCUGUAA.

SEQ ID NO: 31 is a 5′ to 3′ RNA construct from primers T7-SiGNBP-248F / T7-SiGNBP-361R forming one strand of the dsRNA product referred to as dsRNA-SiGNBP-B:

GGGACAAAACAUUGCGUCUUUGGGAUCUCGCAGCAGGUCGUACGACCA
GACGAUUUGAAGACCAUACCAAGGAUGUUUUGAGUGUCGCUUUUUCCG
UGGACAAUCGUCAGAUUG.

SEQ ID NO: 32 is a 5′ to 3′ RNA construct from primers T7-SiGNBP-248F / T7-SiGNBP-361R forming one strand of the dsRNA product referred to as dsRNA-SiGNBP-B, with SEQ ID NO: 32 being complementary to SEQ ID NO: 31:

CAAUCUGACGAUUGUCCACGGAAAAAGCGACACUCAAAACAUCCUUGG
UAUGGUCUUCAAAUCGUCUGGUCGUACGACCUGCUGCGAGAUCCCAAA
GACGCAAUGUUUUGUCCC.

SEQ ID NO: 33 is a 5′ to 3′ RNA construct from primers T7-SiGNBP-361F / T7-SiGNBP-653R forming one strand of the dsRNA product referred to as dsRNA-SiGNBP-C:

CGUGGACAAUCGUCAGAUUGUUUCCGGUUCGCGAGACAAGACAAUUAA
AUUGUGGAAUACAUUGGCCGAAUGCAAGUAUACCAUCCAGGAUGAUGG
GCAUACAGAUUGGGUCAGCUGUGUGCGCUUCUCCCCCAAUCAUGCAAA
UCCCAUCAUUGUCUCUGCAGGCUGGGAUCGCGUUGUCAAGGUCUGGAA
CUUAACGAACUGCAGGUUGAAGAUCAAUCAUAGUGGUCAUACAGGAUA
UCUGAAUACAGUUACGGUAUCGCCUGAUGGAUCACUCUGCGCAUCCGG
UGGAAAAGAUUGCAAAGCUAUGCUGUGGGAUCUGAAUGAUGGA.

SEQ ID NO: 34 is a 5′ to 3′ RNA construct from primers T7-SiGNBP-361F / T7-SiGNBP-653R forming one strand of the dsRNA product referred to as dsRNA-SiGNBP-C, with SEQ ID NO: 34 being complementary to SEQ ID NO: 33:

UCCAUCAUUCAGAUCCCACAGCAUAGCUUUGCAAUCUUUUCCACCGGA
UGCGCAGAGUGAUCCAUCAGGCGAUACCGUAACUGUAUUCAGAUAUCC
UGUAUGACCACUAUGAUUGAUCUUCAACCUGCAGUUCGUUAAGUUCCA
GACCUUGACAACGCGAUCCCAGCCUGCAGAGACAAUGAUGGGAUUUGC
AUGAUUGGGGGAGAAGCGCACACAGCUGACCCAAUCUGUAUGCCCAUC
AUCCUGGAUGGUAUACUUGCAUUCGGCCAAUGUAUUCCACAAUUUAAU
UGUCUUGUCUCGCGAACCGGAAACAAUCUGACGAUUGUCCACG.

SEQ ID NO: 35 is a 5′ to 3′ RNA construct from primers T7-SiGNBP-653F / T7-SiGNBP-940R forming one strand of the dsRNA product referred to as dsRNA-SiGNBP-D:

UGUGGGAUCUGAAUGAUGGAAAACAUCUCCACACUUUAGACCAUAAUG
ACAUCAUUACGGCAUUAUGCUUUAGUCCUAAUCGUUAUUGGCUGUGCG
CCGCAUUCGGACCAUGGAUUAAGAUAUGGGAUCUUGAGACUAAGGAAA
UGGUUGAAGAACUGAAACCUGAGGUUGUGUCUGCAACUAGUAAAGCAG
AGCCACCUCUCUGUUUGUCCCUCGCGUGGUCCACCGACGGUCAAACGU
UGUUCGCUGGAUACUCCGAUAAUACUAUUCGUGUCUGGCAAGUUUCUG.

SEQ ID NO: 36 is a 5′ to 3′ RNA construct from primers T7-SiGNBP-653F / T7-SiGNBP-940R forming one strand of the dsRNA product referred to as dsRNA-SiGNBP-D, with SEQ ID NO: 36 being complementary to SEQ ID NO: 35:

CAGAAACUUGCCAGACACGAAUAGUAUUAUCGGAGUAUCCAGCGAACA
ACGUUUGACCGUCGGUGGACCACGCGAGGGACAAACAGAGAGGUGGCU
CUGCUUUACUAGUUGCAGACACAACCUCAGGUUUCAGUUCUUCAACCA
UUUCCUUAGUCUCAAGAUCCCAUAUCUUAAUCCAUGGUCCGAAUGCGG
CGCACAGCCAAUAACGAUUAGGACUAAAGCAUAAUGCCGUAAUGAUGU
CAUUAUGGUCUAAAGUGUGGAGAUGUUUUCCAUCAUUCAGAUCCCACA.

SEQ ID NO: 37 is a 5′ to 3′ RNA construct from primers T7-SiGNBP-1F / T7-SiGNBP-361R forming one strand of the dsRNA product referred to as dsRNA-SiGNBP-E:

AUGACCGAGACUUUACAGCUGAGAGGGACGCUUCGCGGCCACAAUGGA
UGGGUCACGCAAAUCGCGACAAACCCGAAAUAUCCAGACAUGAUUUUG
UCUUCUUCACGUGAUAAGACUCUGAUUGUGUGGAAAUUGACUCGUGAU
GAAGCUAACUAUGGUAUCCCGCAGAAGCGUCUCUAUGGUCACUCACAC
UUCAUAAGUGAUGUAGUUCUUUCAUCUGAUGGUAACUACGCUCUGUCU
GGUUCAUGGGACAAAACAUUGCGUCUUUGGGAUCUCGCAGCAGGUCGU
ACGACCAGACGAUUUGAAGACCAUACCAAGGAUGUUUUGAGUGUCGCU
UUUUCCGUGGACAAUCGUCAGAUUG.

SEQ ID NO: 38 is a 5′ to 3′ RNA construct from primers T7-SiGNBP-1F / T7-SiGNBP-361R forming one strand of the dsRNA product referred to as dsRNA-SiGNBP-E, with SEQ ID NO: 38 being complementary to SEQ ID NO: 37:

CAAUCUGACGAUUGUCCACGGAAAAAGCGACACUCAAAACAUCCUUGG
UAUGGUCUUCAAAUCGUCUGGUCGUACGACCUGCUGCGAGAUCCCAAA
GACGCAAUGUUUUGUCCCAUGAACCAGACAGAGCGUAGUUACCAUCAG
AUGAAAGAACUACAUCACUUAUGAAGUGUGAGUGACCAUAGAGACGCU
UCUGCGGGAUACCAUAGUUAGCUUCAUCACGAGUCAAUUUCCACACAA
UCAGAGUCUUAUCACGUGAAGAAGACAAAAUCAUGUCUGGAUAUUUCG
GGUUUGUCGCGAUUUGCGUGACCCAUCCAUUGUGGCCGCGAAGCGUCC
CUCUCAGCUGUAAAGUCUCGGUCAU.

SEQ ID NO: 39 is a 5′ to 3′ RNA construct from primers T7-SiGNBP-361F / T7-SiGNBP-940R forming one strand of the dsRNA product referred to as dsRNA-SiGNBP-F:

CGUGGACAAUCGUCAGAUUGUUUCCGGUUCGCGAGACAAGACAAUUAA
AUUGUGGAAUACAUUGGCCGAAUGCAAGUAUACCAUCCAGGAUGAUGG
GCAUACAGAUUGGGUCAGCUGUGUGCGCUUCUCCCCCAAUCAUGCAAA
UCCCAUCAUUGUCUCUGCAGGCUGGGAUCGCGUUGUCAAGGUCUGGAA
CUUAACGAACUGCAGGUUGAAGAUCAAUCAUAGUGGUCAUACAGGAUA
UCUGAAUACAGUUACGGUAUCGCCUGAUGGAUCACUCUGCGCAUCCGG
UGGAAAAGAUUGCAAAGCUAUGCUGUGGGAUCUGAAUGAUGGAAAACA
UCUCCACACUUUAGACCAUAAUGACAUCAUUACGGCAUUAUGCUUUAG
UCCUAAUCGUUAUUGGCUGUGCGCCGCAUUCGGACCAUGGAUUAAGAU
AUGGGAUCUUGAGACUAAGGAAAUGGUUGAAGAACUGAAACCUGAGGU
UGUGUCUGCAACUAGUAAAGCAGAGCCACCUCUCUGUUUGUCCCUCGC
GUGGUCCACCGACGGUCAAACGUUGUUCGCUGGAUACUCCGAUAAUAC
UAUUCGUGUCUGGCAAGUUUCUG.

SEQ ID NO: 40 is a 5′ to 3′ RNA construct from primers T7-SiGNBP-361F / T7-SiGNBP-940R forming one strand of the dsRNA product referred to as dsRNA-SiGNBP-F, with SEQ ID NO: 40 being complementary to SEQ ID NO: 39:

CAGAAACUUGCCAGACACGAAUAGUAUUAUCGGAGUAUCCAGCGAACA
ACGUUUGACCGUCGGUGGACCACGCGAGGGACAAACAGAGAGGUGGCU
CUGCUUUACUAGUUGCAGACACAACCUCAGGUUUCAGUUCUUCAACCA
UUUCCUUAGUCUCAAGAUCCCAUAUCUUAAUCCAUGGUCCGAAUGCGG
CGCACAGCCAAUAACGAUUAGGACUAAAGCAUAAUGCCGUAAUGAUGU
CAUUAUGGUCUAAAGUGUGGAGAUGUUUUCCAUCAUUCAGAUCCCACA
GCAUAGCUUUGCAAUCUUUUCCACCGGAUGCGCAGAGUGAUCCAUCAG
GCGAUACCGUAACUGUAUUCAGAUAUCCUGUAUGACCACUAUGAUUGA
UCUUCAACCUGCAGUUCGUUAAGUUCCAGACCUUGACAACGCGAUCCC
AGCCUGCAGAGACAAUGAUGGGAUUUGCAUGAUUGGGGGAGAAGCGCA
CACAGCUGACCCAAUCUGUAUGCCCAUCAUCCUGGAUGGUAUACUUGC
AUUCGGCCAAUGUAUUCCACAAUUUAAUUGUCUUGUCUCGCGAACCGG
AAACAAUCUGACGAUUGUCCACG.

SEQ ID NO: 41 is T7 promoter sequence TAATACGACTCACTATAGGG.

### DETAILED DESCRIPTION OF THE INVENTION

Disclosed here are specific insect pest dsRNA constructs that target GNBP gene expression. Using dsRNA inhibiting expression of the GNBP gene as a means of interfering with critical functions of the GNBP gene peptide products, a novel method to develop nucleic acid control for pest management is disclosed.

Definitions

As used in the specification and claims, the singular form “a”, “an” and “the” include plural references unless the context clearly dictates otherwise. For example, the term “a cell” includes a plurality of cells, including mixtures thereof.

The term “gene” refers to a DNA sequence involved in producing a polypeptide or precursor thereof. The polypeptide can be encoded by a full-length coding sequence or by any portion of the coding sequence, such as exon sequences. In one embodiment of the invention, the gene target is a GNBP gene of S. invicta.

The term “oligonucleotide” refers to a molecule comprising a plurality of deoxyribonucleotides or ribonucleotides. Oligonucleotide may be generated in any manner, including chemical synthesis, DNA replication, reverse transcription, polymerase chain reaction, or a combination thereof. The present invention embodies utilizing the oligonucleotide in the form of dsRNA as means of interfering with BNBP that leads to control of the target insect. Inasmuch as mononucleotides are synthesized to construct oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide.

The term “primer” refers to an oligonucleotide, which is capable of acting as a point of initiation of synthesis when placed under conditions in which primer extension is initiated. An oligonucleotide “primer” may occur naturally, as in a purified restriction digest or may be produced synthetically.

A primer is selected to be “substantially complementary” to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence is sufficiently complementary with the sequence of the template to hybridize and thereby form a template primer complex for synthesis of the extension product of the primer.

The term “double stranded RNA” or “dsRNA” refers to two substantially complementary strands of ribonucleic acid. “Identity” as used herein, is the relationship between two or more polynucleotide sequences, as determined by comparing the sequences. Identity also means the degree of sequence relatedness between polynucleotide sequences, as determined by the match between strings of such sequences. Identity can be readily calculated (see, .e.g, Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While there exist a number of methods to measure identity between two polynucleotide sequences, the term is well known to skilled artisans (see, e.g., Sequence Analysis in Molecular Biology, von Heinje, G., Academic Press (1987); and Sequence Analysis Primer, Gribskov., M. and Devereux, J., eds., M Stockton Press, New York (1991)). Methods commonly employed to determine identity between sequences include, for example, those disclosed in Carillo, H., and Lipman, D., SIAM J. Applied Math. (1988) 48:1073. “Substantially identical” as used herein, means there is a very high degree of homology (preferably 100% sequence identity) between the inhibitory dsRNA and the corresponding part of the target gene. However, dsRNA having greater than 90% or 95% sequence identity may be used in the present invention, and thus sequence variations that might be expected due to genetic mutation, strain polymorphism, or evolutionary divergence can be tolerated. Although 100% identity is preferred, the dsRNA may contain single or multiple base pair random mismatches between the RNA and the target gene, provided that the mismatches occur at a distance of at least three nucleotides from the fusion site.

As used herein, “target gene” refers to a section of a DNA strand of a double-stranded DNA that is complementary to a section of a DNA strand, including all transcribed regions, that serves as a matrix for transcription. The target gene is therefore usually the sense strand. In one specific embodiment of the invention, the target gene is SEQ ID NO: 7 and fragment thereof.

The term “complementary RNA strand” refers to the strand of the dsRNA, which is complementary to an mRNA transcript that is formed during expression of the target gene, or its processing products. “dsRNA” refers to a ribonucleic acid molecule having a duplex structure comprising two complementary and anti-parallel nucleic acid strands. Not all nucleotides of a dsRNA must exhibit Watson-Crick base pairs. The maximum number of base pairs is the number of nucleotides in the shortest strand of the dsRNA.

“Small interfering RNA” or “siRNA” refers to a short double-strand of ribonucleic acid, approximately 18 to 30 nucleotides in length. The term “RNA interference” or “RNAi” refers to a cellular mechanism for the destruction of targeted ribonucleic acid molecules. Under endogenous conditions, RNAi mechanism operates when dsRNA is cleaved to siRNA via an enzyme, DICER. The siRNA is processed to a single strand of anti-sense ribonucleic acid and coupled with a protein complex named RISC. The antisense RNA then targets a complementary gene construct, such as messenger RNA that is cleaved by ribonuclease. While the examples infra discloses constructing dsRNA constructs via enzymatic techniques with the enzyme RNA polymerase, it is contemplated that siRNA can be constructed via RNA oligonucleotide synthesis such as those disclosed in Scaringe, S., Methods Enzymol., 2000, Vol. 317:3 and incorporated herein by reference.

Disclosed herein are long dsRNA constructs, such as the SEQ ID NOS: 29, 31, 33, 35, 37, and 39. It is contemplated that siRNA and/or partial dsRNA sequences from those sequence listings constructs comprising various double-stranded base pairs of disclosed long dsDNA constructs would be effective in knocking-down the GNBP function in a target ant species. It is contemplated that such siRNA and/or partial dsRNA sequences from SEQ ID NOS: 30, 32, 34, 36, 38 and SEQ ID NO: 40 constructs could be generated synthetically or enzymatically in accordance with the teachings herein.

As used herein, “knock-down” is defined as the act of binding an oligonucleotide with a complementary nucleotide sequence of a gene as such that the expression of the gene or mRNA transcript decreases. In an embodiment, knock-down of a GNBP gene occurs via injection of a dsRNA that can have multiple negative effects on the target insect, such as untimely death of the target ant.

The term “substantially single-stranded” when used in reference to a nucleic acid product means that the product molecule exists primarily as a single strand of nucleic acid in contrast to a double-stranded product which exists as two strands of nucleic acids which are held together by inter-strand base pairing interactions.

“Oligonucleotide primers matching or complementary to a gene sequence” refers to oligonucleotide primers capable of facilitating the template-dependent synthesis of single or double-stranded nucleic acids. Oligonucleotide primers matching or complementary to a gene sequence may be used in PCRs, RT-PCRs and the like.

The term “corresponds to” as used herein means a polynucleotide sequence homologous to all or a portion of a reference polynucleotide sequence, or a polypeptide sequence that is identical to a reference polypeptide sequence. In contradistinction, the term “complementary to” is used herein to mean that the complementary sequence is homologous to all or a portion of a reference polynucleotide sequence. For example, the nucleotide sequence “TATAC” corresponds to a reference sequence “TATAC” and is complementary to a reference sequence “GTATA”.

An “effective amount” is an amount sufficient to effect desired beneficial or deleterious results. An effective amount can be administered in one or more administrations. In terms of treatment, an “effective amount” is that amount sufficient to make the target pest non-functional by causing an adverse effect on that pest, including (but not limited to) physiological damage to the pest; inhibition or modulation of pest growth; inhibition or modulation of pest reproduction; or death of the pest. In one embodiment of the invention, a dsRNA containing solution is fed to a target insect in an amount of approximately at a concentration of 0.20 μg/μl of solution. An effective amount include amounts less that that concentration in which pest mortality would still occur.

The term “solvent” includes any liquid that holds another substance in solution. Examples of solvents include but are not limited to water and organic solvents such as acetone, ethanol, dimethyl sulfoxide (DMSO), and dimethylformamide (DMF).

The term “phagostimulant” refers to any substance that will entice the insect to ingest the dsRNA. For insects, suitable phagostimulants include but are not limited to edible oils and fats, vegetable seed meals, meal by-products such as blood, fish meal, syrups, honey, aqueous solutions of sucrose, artificial sweeteners such as sucralose, saccharin, and other artificial sweeteners, peanut butter, cereals, amino acids, and other proteins.

Methods which are well known to those skilled in the art may be used to construct expression vectors containing sequences encoding Guanine nucleotide binding protein gene and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

While the examples provided wherein describe dsRNA constructs cloned from GenBank Accession No. HM130685 (Solenopsis invicta), it is contemplated that when read in conjunction with the teaching disclosed herein, the construction of other dsRNA constructs targeting GNBP gene sequences of other insect orders would be feasible to those skilled the in the art. Additionally it is contemplated that a single dsRNA construct would be effective in controlling a plurality of insect species.

Statistical analysis used in the foregoing examples is analyzed using the Student's t-test, and t-values and p-values. Comparisons of means were reported when normality and equal variance tests were passed. Significant differences between the data were determined using SigmaPlot software (SigmaPlot®11.2, Systat Software, Inc. San Jose, Calif.).

Insect Colonies used

Solenopsis invicta colonies used in the foregoing Examples were three colonies of the red imported fire ants were collected from Washington County, Miss., in 2009 and 2010. Colonies were kept in an insect growth chamber (27±1 ° C., RH 70±1%, L: D 12: 12) in the National Biological Control Laboratory, Stoneville, Miss. The colonies were fed with 10% sugar and water for seven days before a feeding bioassay. Only workers were used to use the following Examples.

### EXAMPLE 1

### Constructing dsRNA construct for Solenopsis invicta Cloning and sequencing of GNBP gene from Solenopsis invicta

The total RNAs were isolated from Solenopsis invicta workers obtained from the colony descripbed above using TRIzol reagent according to the manufacturer's instructions (Invitrogen, Carlsbad, Calif.). Poly (A)+ RNA was isolated applying Oligotex-dT suspension (QIAGEN, Valencia, Calif.). RNA samples were quantified using a NanoPhotometer™ (IMPLEN, Westlake Village, Calif.).

The GeneRacer™ Kit was used to amplify full-length gene of 5′ and 3′ cDNA ends using modified manufacturer's instruction (Invitrogen, Carlsbad, Calif.). PCR products were cloned using the TOPO TA Cloning® Kit for sequencing (Invitrogen, Carlsbad, Calif.). Transformed plasmids were inserted into One Shot® TOP10 Competent Cells (Invitrogen, Carlsbad, Calif.) and grown overnight on Luria-Bertani plates containing ampicillin and X-Gal (5-bromo-4-chloro-3-indolyl- beta-D-galactopyranoside). Clones were isolated and grown overnight in LB-ampicillin broth at 37 ° C. and 235 RPM in the Innova™ 4000 Incubator Shaker (New Brunswick Scientific, Edison, N.J.).

The plasmids from the GeneRacer library were purified with QIAprep Miniprep (QIAGEN, Valencia, Calif.). The plasmid DNAs (0.5 μg) were then digested by using EcoRI enzyme (2.5 U) for 1.5 h and were run on a 1% agarose gel to confirm the DNA insert. Selected plasmids were then sequenced at the DNA Sequencing Core Laboratory at the Interdisciplinary Center for Biotechnology Research (ICBR), University of Florida (Gainesville, Fla.) and analyzed using the National Center for Biotechnology Information (NCBI) BLASTN program to identify sequence homologies. The sequences were submitted into NCBI GenBank and the accession numbers were HM130684 and HM130685.

### Construction of GNBP dsRNA Constructs

The plasmid DNA (containing full length SiGNBP gene, GenBank Accession Number: HM130685) from the GeneRacer library was diluted as the template using the primers indicated in Tables 1A and 1B. All primers for the synthesis of dsRNA products of SiGNBP were designed based on the sequence of the mRNA (HM130685) and were performed using PRIMER3-Design Primer Pairs and Probes program from Biology Workbench (http://workbench.sdsc.edu). The dsRNA products (dsRNA-SiGNBP-A, dsRNA-SiGNBP-B, dsRNA-SiGNBP-C, dsRNA-SiGNBP-D, dsRNA-SiGNBP-E, and dsRNA-SiGNBP-F) were designed to cover the both end portion of SiGNBP, containing 255, 114, 331, 288, 361, and 599 by gene fragments respectively, with the T7-SiGNBP-13F/T7-SiGNBP-267R, T7-SiGNBP-248F/T7-SiGNBP-361R, T7-SiGNBP-361F/T7-SiGNBP-653R, T7-SiGNBP-653F/T7-SiGNBP-940R, T7-SiGNBP-1F/T7-SiGNBP-361R, and T7-SiGNBP-361F/T7-SiGNBP-619R primers (Table 1A) in which T7 promoter sequence (TAATACGACTCACTATAGGG) (SEQ ID NO: 41) added to the 5′ end of each primer. PCR condition were 95 ° C. for 4 min, followed by 36 cycles of 95 ° C. 30 s, 55 ° C. for 30 s and 72 ° C. for 1.5 min, finishing with an extension step at 72 ° C. for 10 min. PCR products were purified using a QIAquick PCR purification kit (QIAGEN, Valencia, CA). The six resulting templates were then transcribed for 4 hours using T7 RNA polymerase following the manufacturer's protocol of the MEGAscript® RNAi Kit (Ambion, Austin, Tex.). DNA and single stranded RNA were removed and the dsRNA products were then purified following the manufacturer's protocol. The quality of the dsRNA was determined by electrophoresis and quantified using a NanoPhotometer™ (IMPLEN, Westlake Village, Calif.).

TABLE 1A
Primers used for the synthesis of dsRNA products.
Primer Name Sequence

T7-SiGNBP-1F (SEQ ID NO: 14):
TAATACGACTCACTATAGGGATGACCGAGACTTTACAGCT
T7-SiGNBP-267R (SEQ ID NO: 15):
TAATACGACTCACTATAGGGAAGACGCAATGTTTTGTCCC
T7-SiGNBP-653F (SEQ ID NO: 16):
TAATACGACTCACTATAGGGTGTGGGATCTGAATGATGGA
T7-SiGNBP-940R (SEQ ID NO: 17):
TAATACGACTCACTATAGGGCAGAAACTTGCCAGACACGA
T7-SiGNBP-13F (SEQ ID NO: 18):
TAATACGACTCACTATAGGGTTACAGCTGAGAGGGACGCT
T7-SiGNBP-248F (SEQ ID NO: 19):
TAATACGACTCACTATAGGGGGGACAAAACATTGCGTCTT
T7-SiGNBP-361R (SEQ ID NO: 20):
TAATACGACTCACTATAGGGCAATCTGACGATTGTCCACG
T7-SiGNBP-361F (SEQ ID NO: 21):
TAATACGACTCACTATAGGGCGTGGACAATCGTCAGATTG
T7-SiGNBP-653R (SEQ ID NO: 22):
TAATACGACTCACTATAGGGTCCATCATTCAGATCCCACA

TABLE 1B
The sizes of dsRNA products

| dsRNA Name | Primers | Products size |
|---|---|---|
| dsRNA-SiGNBP-A | T7-SiGNBP-13F/T7-SiGNBP- | 255 bp |
| (SEQ ID NO: 29, 30) | 267R | |
| dsRNA-SiGNBP-B | T7-SiGNBP-248F/T7- | 114 bp |
| (SEQ ID NO: 31, 32) | SiGNBP-361R | |
| dsRNA-SiGNBP-C | T7-SiGNBP-361F/T7- | 331 bp |
| (SEQ ID NO: 33, 34) | SiGNBP-653R | |
| dsRNA-SiGNBP-D | T7-SiGNBP-653F/T7- | 288 bp |
| (SEQ ID NO: 35, 36) | SiGNBP-940R | |
| dsRNA-SiGNBP-E | T7-SiGNBP-1F/T7-SiGNBP- | 361 bp |
| (SEQ ID NO: 37, 38) | 361R | |
| dsRNA-SiGNBP-F | T7-SiGNBP-361F/T7- | 599 bp |
| (SEQ ID NO: 39, 40) | SiGNBP-940R | |

### EXAMPLE 2

### dsRNA construct feeding bioassay using liquid bait station

To determine the toxicity of dsRNA-GNBP products of Table 1B, each dsRNA product was diluted in 10% sugar solution. A 700 μl glass conical insert (8×40 mm) was mounted on an inverted Petri dish using a snap cap of 1.5-ml disposable conical economy micro tube (VWR, West Chester, Pa., USA). The snap cap was first attached upside down at the center of Petri dish (60×15 mm) using glue (Arrow Fastener Co., Inc., Saddle Brook, N.J.). A 4-mm diameter hole was then drilled at the center, which went through the Petri dish and the snap cap. After the insert was filled with test liquid, the top part of the insert was wrapped with a small piece of Teflon tape and a 9-mm disk of micronic mesh (400 meshes) was then placed on the top of the insert. The insert was then pushed into the snap cap. The micronic mesh not only provided ants with a clean and uniform feeding arena, but also prevented leaking of the test liquid from the insert. There was an entrance (a half circle of 4 mm diameter) on the edge of the inverted Petri dish which allowed ants to have access to the feeding arena. The feeding station was placed in a larger Petri dish (100×25 mm) with the inner wall coated with Fluon® (Ag Fluoropolymers, Chadds Ford, Pa.) to prevent escape. Two-hundred mg of worker ants (˜200±20 individual) were introduced into the large Petrie dish. Three colonies were used for the bioassay. In the treatment, the test liquid contained dsRNA-SiGNBP in 10% sugar solution. In the control, ants were provided with 10% sugar solution only. During the bioassay, the ants were maintained in an insect growth chamber (27±1° C., RH 70±1%, L: D=12:12). Mortality was recorded after 12, 24, 48, 72, and 96 h. Samples were collected after 12 h for testing mRNA expression knock-down. The samples were stored in a −80 ° C. freezer before RNA extraction. The feeding bioassay was repeated three times.

Our RNAi experiment showed that the SiGNBP mRNA levels decreased by 40%-90% after 12 h feeding of dsRNA-SiGNBP, indicating that this gene was successfully silenced by RNAi. The different constructs of dsRNA-SiGNBP revealed that the different parts of the gene and fragment length may affect gene expression. To determine whether dsRNA-SiGNBP products affect SiGNBP gene expression, qPCR was used to examine the gene function during feeding.

qPCR of Worker Ants fed dsRNA construct

For qPCR analysis of worker ants fed dsRNA constructs for the feeding assay, total RNA from the ants were extracted using TRIzol reagent according to the manufacturer's instructions as mentioned above on RNA extraction. To ensure that no genomic DNA contaminated the sample, Oligotex mRNA mini Kit (QIAGEN, Valencia, Calif.) was used to purify total RNA. A 200 ng aliquot of purified mRNA was reversely transcribed in a 20-μl reaction volume using Clone AMV first-Strand Synthesis Kit and Oligo (dT) primer for cDNA synthesis according to the manufacturer's instructions (Invitrogen, Carlsbad, Calif.). Oligo dT primer was used to synthesize the first strand cDNA library. The RT-PCR reaction was conducted at 42 ° C. for 3 h. The reaction was terminated by heat inactivation at 95 ° C. for 5 min. The cDNA samples for dsRNA treatment and controls were diluted by adding 80 μl ddH2O (˜450±100 ng/μl) and stored at −20 ° C.

To design gene-specific primers, detailed analyses of the nucleotide sequence of genes (Genbank Accession Number: HM130685) were performed using the PRIMER3-Design Primer Pairs and Probes program from Biology Workbench (http://workbench.sdsc.edu). The primers for the S. invicta actin gene (Genbank Accession Number: HM130684) were also designed for internal control and comparison.

The qPCR assay for SiGNBP gene was performed using Platinum® SYBR® Green qPCR SuperMix-UDG with ROX (Invitrogen, Carlsbad, Calif.) in a volume of 15 μl on an Applied Biosystems 7300 Fast Real-Time PCR System (Foster City, Calif.). The PCR mixture consisted of 1 μl diluted cDNA (˜300 ng/μl), 0.5 μM primers and 1× master mix. In every qPCR run, actin was used as an internal control to normalize for variation in the amount of cDNA template. The qPCR primers for actin gene were SiActin-783-F 5′- CCTCTTCCAACCTTCCTTCC-3′ (SEQ ID No: 23) and SiActin-948R 5′- CTTTTGCATACGATCAGCGA-3′ (SEQ ID No: 24) (Table 2). The qPCR thermal cycling parameters were 50 ° C. for 2 min, 95 ° C. for 10 min followed by 40 cycles of 95 ° C. for 15 s and 60 ° C. for 1 min. This was followed by the dissociation stage at 95 ° C. for 15 s, 60 ° C. for 30 s and 95 ° C. for 15 s. Relative expression levels were calculated as follows. First, SiGNBP transcript levels relative to a standard (SiACTIN) were calculated using the formula ΔCT=CT(SiGNBP)−CT(SiACTIN). Second, an average ACT value for each sample was calculated. Third, relative expression levels were calculated using the equation 1000×2^{[-average ΔCT]}.

TABLE 2
Primers used for quantitative real-time PCR.
Primer Name Sequence

SiActin-605F GGTCGCGATCTTACCGATTA (SEQ ID NO: 25)
SiActin-859R GAAGGAAGGTTGGAAGAGGG (SEQ ID NO: 26)
SiGNBP-13F TTACAGCTGAGAGGGACGCT (SEQ ID NO: 27)
SiGNBP-267R AAGACGCAATGTTTTGTCCC (SEQ ID NO: 28)

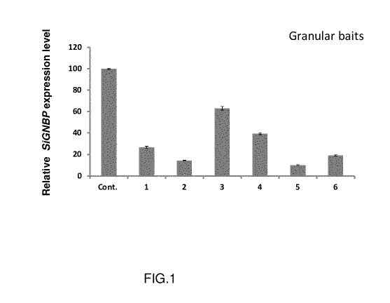

After 12 h of feeding on dsRNA-SiGNBP, the relative gene expressions of SiGNBP were down-regulated 73.36±1.04%, 85.59±1.25%, 36.89±1.75%, 60.54±0.89%, 90.02 ±0.51%, and 80.95±0.58% bydsRNA-SiGNBP-A, dsRNA-SiGNBP-B, dsRNA-SiGNBP-C, dsRNA-SiGNBP-D, dsRNA-SiGNBP-E, and dsRNA-SiGNBP-F, respectively (FIG. 1).

Ant mortality occurred after 12 h (Table 3). dsRNA-SiGNBP-A, dsRNA-SiGNBP-B, dsRNA-SiGNBP-C, dsRNA-SiGNBP-D, dsRNA-SiGNBP-E, and dsRNA-SiGNBP-F caused 5.1 ±2.7%, 5.2±1.6%, 7.8±3.6%, 6.4±3.4%, 6.8±1.6%, and 8.4±2.0% mortality respectively. After 24 h, mortality was 8.9-18.6% (i.e., dsRNA-SiGNBP-A, 8.9±3.5%; dsRNA-SiGNBP-B, 9.5±3.8%; dsRNA-SiGNBP-C, 18.6±10.6%; dsRNA-SiGNBP-D, 13.8±8.8%; dsRNA-SiGNBP-E, 14.1±3.5%; and dsRNA-SiGNBP-F, 18.1±6.9%). After 48 h, mortality reached 44.2-69.7%. After 72 h and 96 h, mortality reached 85.1-96.2%.

TABLE 3
Toxicities of dsRNA-SiGNBP products against workers of
Solenopsis invicta using liquid station feeding.

| | dsRNA | | | | | |
|---|---|---|---|---|---|---|
| | concentra- | Mortality (%) Average ± SD | | | | |
| Solution | tion (μg/μl) | 15 h | 24 h | 48 h | 72 h | 96 h |
| 10% sugar solution a | — | 1.2 ± 1.1 | 3.0 ± 1.1 | 2.2 ± 1.03 | 5.2 ± 1.6 | 15.7 ± 7.7 |
| dsRNA-SiGNBP-A b | 0.200 | 5.1 ± 2.7 | 8.9 ± 3.5 | 57.3 ± 6.6 | 89.1 ± 6.5 | 94.8 ± 8.5 |
| dsRNA-SiGNBP-B b | 0.200 | 5.2 ± 1.6 | 9.5 ± 3.8 | 44.2 ± 10.8 | 85.1 ± 7.1 | 87.8 ± 7.0 |
| dsRNA-SiGNBP-C b | 0.200 | 7.8 ± 3.6 | 18.6 ± 10.6 | 68.2 ± 12.8 | 91.5 ± 4.4 | 95.8 ± 4.1 |
| dsRNA-SiGNBP-D b | 0.200 | 6.4 ± 3.4 | 13.8 ± 8.8 | 67.3 ± 24.8 | 91.3 ± 4.5 | 96.2 ± 4.5 |
| dsRNA-SiGNBP-E b | 0.200 | 6.8 ± 1.6 | 14.1 ± 3.5 | 69.7 ± 10.3 | 89.8 ± 2.8 | 94.8 ± 2.6 |
| dsRNA-SiGNBP-F b | 0.200 | 8.4 ± 2.0 | 18.1 ± 6.9 | 66.3 ± 12.2 | 85.8 ± 4.7 | 94.9 ± 2.5 |

a Control without dsRNA.
b 10% sugar solution and dsRNA

### EXAMPLE 3

### dsRNA construct feeding bioassay using granular bait

The most cost-effective method for managing fire ants over a large area is to broadcast insecticidal bait. In contrast to a liquid bait station, a broadcasting application requires that the active ingredient be formulated into a carrier which can be broadcasted. The most common fire ant bait carrier on the current market is corn grit. However, corn grit is very sensitive to water and thus cannot be used to formulate water-based bait such as a dsRNA solution. A water resistant fire ant granular carrier was recently developed at the Biological Control of Pests Research Unit, USDA-ARS, Stoneville, MS. The toxicity of dsRNAs using that granular carrier was determined in this study.

Each dsRNA product was diluted in a 10% sugar solution with a dsRNA concentration of 200 ng/μl (Table 4). For each dsRNA-GNBP construct, 200 μl of solution was mixed with 200 mg of the bait carrier. Bait was placed in a cap of a Wheaton 20-ml glass scintillation vial and then placed at the center of a plastic Petri dish (100×25 mm). The inner wall of the Petri dish was coated with Fluon®. The cap with bait was covered with another inverted Petri dish (60×15 mm) which had an entrance hole at the edge. Again, 200 mg of worker ants was used. The concentrations of dsRNA-SiGNBP are shown in Table 4. In the control, 200 μl of a 10% sugar solution was mixed with the carrier. The schedule of sampling, number of replicates, qPCR determination, and ant maintenance were the same as described for the previous bioassay using liquid feeding station in Example 2.

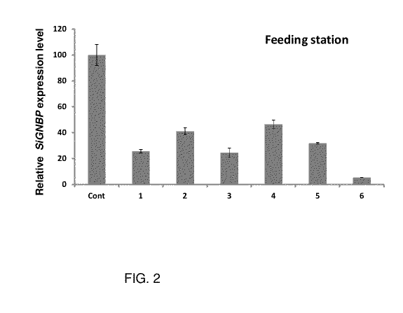

After 12 h feeding on dsRNA granular bait, the relative gene expressions of SiGNBP were all down-regulated with dsRNA-SiGNBP-A having 74.36±1.36% down regulation; dsRNA-SiGNBP-B 58.85±1.25%; dsRNA-SiGNBP-C 75.55±1.75%; dsRNA-SiGNBP-D 53.63±0.89%; dsRNA-SiGNBP-E 68.18±0.51%; and dsRNA-SiGNBP-F 94.66±0.58% (FIG. 2).

As in the liquid feeding station bioassay, mortality occurred after 12 h (Table 4). The dsRNA-SiGNBP-A, dsRNA-SiGNBP-B, dsRNA-SiGNBP-C, dsRNA-SiGNBP-D, dsRNA-SiGNBP-E, and dsRNA-SiGNBP-F caused 9.3±2.6%, 9.1±2.7%, 9.4±4.2%, 6.4±2.5%, 9.5 ±2.8%, and 8.6±3.1% mortality respectively. After 24 h, mortality was 11.3-29.7% (i.e., dsRNA-SiGNBP-A, 21.5±4.5%; dsRNA-SiGNBP-B, 29.7±9.9%; dsRNA-SiGNBP-C, 22.8±11.5%; dsRNA-SiGNBP-D, 14.8±4.1%; dsRNA-SiGNBP-E, 20.8±9.2%; and dsRNA-SiGNBP-F, 11.3±2.9%). After 48 h, mortality reached 60.8-73.2%. After 72 h and 96 h, mortality reached 85.3-97.3%.

TABLE 4
Toxicities of dsRNA-SiGNBP products against workers of
Solenopsis invicta in bioassay using granular bait

| | dsRNA | | | | | |
|---|---|---|---|---|---|---|
| | concentra- | Mortality (%) Average ± SD | | | | |
| Solution | tion (μg/μl) | 12 h | 24 | 48 h | 72 h | 96 h |
| 10% sugar solution a | — | 3.6 ± 1.1 | 6.5 ± 3.6 | 8.8 ± 4.1 | 10.8 ± 5.2 | 17.7 ± 5.8 |
| dsRNA-SiGNBP-A b | 0.200 | 9.3 ± 2.6 | 21.5 ± 4.5 | 62.2 ± 18.2 | 85.3 ± 8.1 | 94.7 ± 4.0 |
| dsRNA-SiGNBP-B b | 0.200 | 9.1 ± 2.7 | 29.7 ± 9.9 | 73.2 ± 9.9 | 86.8 ± 9.0 | 95.3 ± 4.6 |
| dsRNA-SiGNBP-C b | 0.200 | 9.4 ± 4.2 | 22.8 ± 11.5 | 66.8 ± 11.0 | 86.7 ± 7.2 | 95.8 ± 4.1 |
| dsRNA-SiGNBP-D b | 0.200 | 6.4 ± 2.5 | 14.8 ± 4.1 | 72.4 ± 9.1 | 90.8 ± 4.4 | 97.3 ± 3.8 |
| dsRNA-SiGNBP-E b | 0.200 | 9.5 ± 2.8 | 20.8 ± 9.2 | 67.4 ± 20.9 | 86.8 ± 7.8 | 96.7 ± 2.1 |
| dsRNA-SiGNBP-F b | 0.200 | 8.6 ± 3.1 | 11.3 ± 2.9 | 60.8 ± 17.7 | 87.8 ± 6.3 | 95.7 ± 3.2 |

a Control without dsRNA.
b 10% sugar solution and dsRNA.

While the invention has been described with reference to details of the illustrated embodiment, these details are not intended to limit the scope of the invention as defined in the appended claims. The embodiment of the invention in which exclusive property or privilege is claimed is defined as follows:

1. A double-stranded ribonucleic acid (dsRNA) for inhibiting the expression of a guanine nucleotide binding protein in a cell, wherein said dsRNA comprises SEQ ID NO: 29 and an antisense strand comprising a sequence complementary to SEQ ID NO: 29.

2. The double-stranded ribonucleic acid of claim 1 wherein the antisense strand is SEQ ID NO 30.

3. A double-stranded ribonucleic acid (dsRNA) for inhibiting the expression of a guanine nucleotide binding protein in a cell, wherein said dsRNA comprises SEQ ID NO: 31 and an antisense strand comprising a sequence complementary to SEQ ID NO: 31.

4. The double-stranded ribonucleic acid of claim 3 wherein the antisense strand is SEQ ID NO 32.

5. A double-stranded ribonucleic acid (dsRNA) for inhibiting the expression of a guanine nucleotide binding protein in a cell, wherein said dsRNA comprises SEQ ID NO: 33 and an antisense strand comprising a sequence complementary to SEQ ID NO: 33.

6. The double-stranded ribonucleic acid of claim 5 wherein the antisense strand is SEQ ID NO 34.

7. A double-stranded ribonucleic acid (dsRNA) for inhibiting the expression of a guanine nucleotide binding protein in a cell, wherein said dsRNA comprises SEQ ID NO: 35 and an antisense strand comprising a sequence complementary to SEQ ID NO: 35.

8. The double-stranded ribonucleic acid of claim 7 wherein the antisense strand is SEQ ID NO 36.

9. A double-stranded ribonucleic acid (dsRNA) for inhibiting the expression of a guanine nucleotide binding protein in a cell, wherein said dsRNA comprises SEQ ID NO: 37 and an antisense strand comprising a sequence complementary to SEQ ID NO: 37.

10. The double-stranded ribonucleic acid of claim 9 wherein the antisense strand is SEQ ID NO 38.

11. A double-stranded ribonucleic acid (dsRNA) for inhibiting the expression of a guanine nucleotide binding protein in a cell, wherein said dsRNA comprises SEQ ID NO: 39 and an antisense strand comprising a sequence complementary to SEQ ID NO: 39.

12. The double-stranded ribonucleic acid of claim 11 wherein the antisense strand is SEQ ID NO 40.

13. A method for controlling Solenopsis invicta, the method comprising: constructing a double stranded ribonucleic acid construct that is complementary to a gene that encodes a guanine nucleotide binding protein, dissolving the double stranded ribonucleic acid to form a solution, and contacting an effective amount of said solution to Solenopsis invicta, wherein said solution is ingested by Solenopsis invicta and RNA interference is induced, resulting in mortality of Solenopsis invica.

14. The method of claim 13, wherein one strand of the double stranded ribonucleic acid is complementary to the nucleotide sequence and selected from the group consisting of SEQ ID NO: 29, SEQ ID NO: 31, SEQ ID NO: 33, SEQ ID NO: 35, SEQ ID NO: 37, and SEQ ID NO: 39.

15. The method of claim 13, wherein the double stranded ribonucleic acid construct is dissolved in a sucrose solution.

16. The method of claim 13, wherein the double stranded ribonucleic acid construct is dissolved in water.

17. The method of claim 13, wherein the solution is applied to Solenopsis invicta bait material.

18. The method of claim 17, wherein the bait material is a granular bait.

19. The Solenopsis invicta control solution constructed by the method of claim 11.

20. The method of claim 7 wherein the effective amount of double stranded ribonucleic acid is approximately 0.20 μg per μl.


SEQUENCE LISTING (41 sequences)

SEQ ID NO: 1 - DNA, 255 bp, Artificial Sequence
  cDNA construct
ttacagctgagagggacgcttcgcggccacaatggatgggtcacgcaaatcgcgacaaacccgaaatatccagacatgattttgtcttcttcacgtgataagactctgattgtgtggaaattgactcgtgatgaagctaactatggtatcccgcagaagcgtctctatggtcactcacacttcataagtgatgtagttctttcatctgatggtaactacgctctgtctggttcatgggacaaaacattgcgtctt

SEQ ID NO: 2 - DNA, 114 bp, Artificial Sequence
  cDNA construct
gggacaaaacattgcgtctttgggatctcgcagcaggtcgtacgaccagacgatttgaagaccataccaaggatgttttgagtgtcgctttttccgtggacaatcgtcagattg

SEQ ID NO: 3 - DNA, 331 bp, Artificial Sequence
  cDNA construct
cgtggacaatcgtcagattgtttccggttcgcgagacaagacaattaaattgtggaatacattggccgaatgcaagtataccatccaggatgatgggcatacagattgggtcagctgtgtgcgcttctcccccaatcatgcaaatcccatcattgtctctgcaggctgggatcgcgttgtcaaggtctggaacttaacgaactgcaggttgaagatcaatcatagtggtcatacaggatatctgaatacagttacggtatcgcctgatggatcactctgcgcatccggtggaaaagattgcaaagctatgctgtgggatctgaatgatgga

SEQ ID NO: 4 - DNA, 288 bp, Artificial Sequence
  cDNA construct
tgtgggatctgaatgatggaaaacatctccacactttagaccataatgacatcattacggcattatgctttagtcctaatcgttattggctgtgcgccgcattcggaccatggattaagatatgggatcttgagactaaggaaatggttgaagaactgaaacctgaggttgtgtctgcaactagtaaagcagagccacctctctgtttgtccctcgcgtggtccaccgacggtcaaacgttgttcgctggatactccgataatactattcgtgtctggcaagtttctg

SEQ ID NO: 5 - DNA, 361 bp, Artificial Sequence
  cDNA construct
atgaccgagactttacagctgagagggacgcttcgcggccacaatggatgggtcacgcaaatcgcgacaaacccgaaatatccagacatgattttgtcttcttcacgtgataagactctgattgtgtggaaattgactcgtgatgaagctaactatggtatcccgcagaagcgtctctatggtcactcacacttcataagtgatgtagttctttcatctgatggtaactacgctctgtctggttcatgggacaaaacattgcgtctttgggatctcgcagcaggtcgtacgaccagacgatttgaagaccataccaaggatgttttgagtgtcgctttttccgtggacaatcgtcagattg

SEQ ID NO: 6 - DNA, 599 bp, Artificial Sequence
  cDNA construct
cgtggacaatcgtcagattgtttccggttcgcgagacaagacaattaaattgtggaatacattggccgaatgcaagtataccatccaggatgatgggcatacagattgggtcagctgtgtgcgcttctcccccaatcatgcaaatcccatcattgtctctgcaggctgggatcgcgttgtcaaggtctggaacttaacgaactgcaggttgaagatcaatcatagtggtcatacaggatatctgaatacagttacggtatcgcctgatggatcactctgcgcatccggtggaaaagattgcaaagctatgctgtgggatctgaatgatggaaaacatctccacactttagaccataatgacatcattacggcattatgctttagtcctaatcgttattggctgtgcgccgcattcggaccatggattaagatatgggatcttgagactaaggaaatggttgaagaactgaaacctgaggttgtgtctgcaactagtaaagcagagccacctctctgtttgtccctcgcgtggtccaccgacggtcaaacgttgttcgctggatactccgataatactattcgtgtctggcaagtttctg

SEQ ID NO: 7 - DNA, 954 bp, Solenopsis invicta
atgaccgagactttacagctgagagggacgcttcgcggccacaatggatgggtcacgcaaatcgcgacaaacccgaaatatccagacatgattttgtcttcttcacgtgataagactctgattgtgtggaaattgactcgtgatgaagctaactatggtatcccgcagaagcgtctctatggtcactcacacttcataagtgatgtagttctttcatctgatggtaactacgctctgtctggttcatgggacaaaacattgcgtctttgggatctcgcagcaggtcgtacgaccagacgatttgaagaccataccaaggatgttttgagtgtcgctttttccgtggacaatcgtcagattgtttccggttcgcgagacaagacaattaaattgtggaatacattggccgaatgcaagtataccatccaggatgatgggcatacagattgggtcagctgtgtgcgcttctcccccaatcatgcaaatcccatcattgtctctgcaggctgggatcgcgttgtcaaggtctggaacttaacgaactgcaggttgaagatcaatcatagtggtcatacaggatatctgaatacagttacggtatcgcctgatggatcactctgcgcatccggtggaaaagattgcaaagctatgctgtgggatctgaatgatggaaaacatctccacactttagaccataatgacatcattacggcattatgctttagtcctaatcgttattggctgtgcgccgcattcggaccatggattaagatatgggatcttgagactaaggaaatggttgaagaactgaaacctgaggttgtgtctgcaactagtaaagcagagccacctctctgtttgtccctcgcgtggtccaccgacggtcaaacgttgttcgctggatactccgataatactattcgtgtctggcaagtttctgtatccagcagataa

SEQ ID NO: 8 - DNA, 255 bp, Artificial Sequence
  cDNA construct
aagacgcaatgttttgtcccatgaaccagacagagcgtagttaccatcagatgaaagaactacatcacttatgaagtgtgagtgaccatagagacgcttctgcgggataccatagttagcttcatcacgagtcaatttccacacaatcagagtcttatcacgtgaagaagacaaaatcatgtctggatatttcgggtttgtcgcgatttgcgtgacccatccattgtggccgcgaagcgtccctctcagctgtaa

SEQ ID NO: 9 - DNA, 114 bp, Artificial Sequence
  cDNA construct
caatctgacgattgtccacggaaaaagcgacactcaaaacatccttggtatggtcttcaaatcgtctggtcgtacgacctgctgcgagatcccaaagacgcaatgttttgtccc

SEQ ID NO: 10 - DNA, 331 bp, Artificial Sequence
  cDNA construct
tccatcattcagatcccacagcatagctttgcaatcttttccaccggatgcgcagagtgatccatcaggcgataccgtaactgtattcagatatcctgtatgaccactatgattgatcttcaacctgcagttcgttaagttccagaccttgacaacgcgatcccagcctgcagagacaatgatgggatttgcatgattgggggagaagcgcacacagctgacccaatctgtatgcccatcatcctggatggtatacttgcattcggccaatgtattccacaatttaattgtcttgtctcgcgaaccggaaacaatctgacgattgtccacg

SEQ ID NO: 11 - DNA, 288 bp, Artificial Sequence
  cDNA construct
cagaaacttgccagacacgaatagtattatcggagtatccagcgaacaacgtttgaccgtcggtggaccacgcgagggacaaacagagaggtggctctgctttactagttgcagacacaacctcaggtttcagttcttcaaccatttccttagtctcaagatcccatatcttaatccatggtccgaatgcggcgcacagccaataacgattaggactaaagcataatgccgtaatgatgtcattatggtctaaagtgtggagatgttttccatcattcagatcccaca

SEQ ID NO: 12 - DNA, 361 bp, Artificial Sequence
  cDNA construct
caatctgacgattgtccacggaaaaagcgacactcaaaacatccttggtatggtcttcaaatcgtctggtcgtacgacctgctgcgagatcccaaagacgcaatgttttgtcccatgaaccagacagagcgtagttaccatcagatgaaagaactacatcacttatgaagtgtgagtgaccatagagacgcttctgcgggataccatagttagcttcatcacgagtcaatttccacacaatcagagtcttatcacgtgaagaagacaaaatcatgtctggatatttcgggtttgtcgcgatttgcgtgacccatccattgtggccgcgaagcgtccctctcagctgtaaagtctcggtcat

SEQ ID NO: 13 - DNA, 599 bp, Artificial Sequence
  cDNA construct
cagaaacttgccagacacgaatagtattatcggagtatccagcgaacaacgtttgaccgtcggtggaccacgcgagggacaaacagagaggtggctctgctttactagttgcagacacaacctcaggtttcagttcttcaaccatttccttagtctcaagatcccatatcttaatccatggtccgaatgcggcgcacagccaataacgattaggactaaagcataatgccgtaatgatgtcattatggtctaaagtgtggagatgttttccatcattcagatcccacagcatagctttgcaatcttttccaccggatgcgcagagtgatccatcaggcgataccgtaactgtattcagatatcctgtatgaccactatgattgatcttcaacctgcagttcgttaagttccagaccttgacaacgcgatcccagcctgcagagacaatgatgggatttgcatgattgggggagaagcgcacacagctgacccaatctgtatgcccatcatcctggatggtatacttgcattcggccaatgtattccacaatttaattgtcttgtctcgcgaaccggaaacaatctgacgattgtccacg

SEQ ID NO: 14 - DNA, 40 bp, Artificial Sequence
  Primer
taatacgactcactatagggatgaccgagactttacagct

SEQ ID NO: 15 - DNA, 40 bp, Artificial Sequence
  Primer
taatacgactcactatagggaagacgcaatgttttgtccc

SEQ ID NO: 16 - DNA, 40 bp, Artificial Sequence
  Primer
taatacgactcactatagggtgtgggatctgaatgatgga

SEQ ID NO: 17 - DNA, 40 bp, Artificial Sequence
  Primer
taatacgactcactatagggcagaaacttgccagacacga

SEQ ID NO: 18 - DNA, 40 bp, Artificial Sequence
  Primer
taatacgactcactatagggttacagctgagagggacgct

SEQ ID NO: 19 - DNA, 40 bp, Artificial Sequence
  Primer
taatacgactcactataggggggacaaaacattgcgtctt

SEQ ID NO: 20 - DNA, 40 bp, Artificial Sequence
  Primer
taatacgactcactatagggcaatctgacgattgtccacg

SEQ ID NO: 21 - DNA, 40 bp, Artificial Sequence
  Primer
taatacgactcactatagggcgtggacaatcgtcagattg

SEQ ID NO: 22 - DNA, 40 bp, Artificial Sequence
  Primer
taatacgactcactatagggtccatcattcagatcccaca

SEQ ID NO: 23 - DNA, 20 bp, Artificial Sequence
  Primer
cctcttccaaccttccttcc

SEQ ID NO: 24 - DNA, 20 bp, Artificial Sequence
  Primer
cttttgcatacgatcagcga

SEQ ID NO: 25 - DNA, 20 bp, Artificial Sequence
  Primer
ggtcgcgatcttaccgatta

SEQ ID NO: 26 - DNA, 20 bp, Artificial Sequence
  Primer
gaaggaaggttggaagaggg

SEQ ID NO: 27 - DNA, 20 bp, Artificial Sequence
  Primer
ttacagctgagagggacgct

SEQ ID NO: 28 - DNA, 20 bp, Artificial Sequence
  Primer
aagacgcaatgttttgtccc

SEQ ID NO: 29 - RNA, 255 nt, Artificial Sequence
  dsRNA-SiGNBP-A
uuacagcugagagggacgcuucgcggccacaauggaugggucacgcaaaucgcgacaaacccgaaauauccagacaugauuuugucuucuucacgugauaagacucugauuguguggaaauugacucgugaugaagcuaacuaugguaucccgcagaagcgucucuauggucacucacacuucauaagugauguaguucuuucaucugaugguaacuacgcucugucugguucaugggacaaaacauugcgucuu

SEQ ID NO: 30 - RNA, 255 nt, Artificial Sequence
  dsRNA-SiGNBP-A
aagacgcaauguuuugucccaugaaccagacagagcguaguuaccaucagaugaaagaacuacaucacuuaugaagugugagugaccauagagacgcuucugcgggauaccauaguuagcuucaucacgagucaauuuccacacaaucagagucuuaucacgugaagaagacaaaaucaugucuggauauuucggguuugucgcgauuugcgugacccauccauuguggccgcgaagcgucccucucagcuguaa

SEQ ID NO: 31 - RNA, 114 nt, Artificial Sequence
  dsRNA-SiGNBP-B
gggacaaaacauugcgucuuugggaucucgcagcaggucguacgaccagacgauuugaagaccauaccaaggauguuuugagugucgcuuuuuccguggacaaucgucagauug

SEQ ID NO: 32 - RNA, 114 nt, Artificial Sequence
  dsRNA-SiGNBP-B
caaucugacgauuguccacggaaaaagcgacacucaaaacauccuugguauggucuucaaaucgucuggucguacgaccugcugcgagaucccaaagacgcaauguuuuguccc

SEQ ID NO: 33 - RNA, 331 nt, Artificial Sequence
  dsRNA-SiGNBP-C
cguggacaaucgucagauuguuuccgguucgcgagacaagacaauuaaauuguggaauacauuggccgaaugcaaguauaccauccaggaugaugggcauacagauugggucagcugugugcgcuucucccccaaucaugcaaaucccaucauugucucugcaggcugggaucgcguugucaaggucuggaacuuaacgaacugcagguugaagaucaaucauaguggucauacaggauaucugaauacaguuacgguaucgccugauggaucacucugcgcauccgguggaaaagauugcaaagcuaugcugugggaucugaaugaugga

SEQ ID NO: 34 - RNA, 331 nt, Artificial Sequence
  dsRNA-SiGNBP-C
uccaucauucagaucccacagcauagcuuugcaaucuuuuccaccggaugcgcagagugauccaucaggcgauaccguaacuguauucagauauccuguaugaccacuaugauugaucuucaaccugcaguucguuaaguuccagaccuugacaacgcgaucccagccugcagagacaaugaugggauuugcaugauugggggagaagcgcacacagcugacccaaucuguaugcccaucauccuggaugguauacuugcauucggccaauguauuccacaauuuaauugucuugucucgcgaaccggaaacaaucugacgauuguccacg

SEQ ID NO: 35 - RNA, 288 nt, Artificial Sequence
  dsRNA-SiGNBP-D
ugugggaucugaaugauggaaaacaucuccacacuuuagaccauaaugacaucauuacggcauuaugcuuuaguccuaaucguuauuggcugugcgccgcauucggaccauggauuaagauaugggaucuugagacuaaggaaaugguugaagaacugaaaccugagguugugucugcaacuaguaaagcagagccaccucucuguuugucccucgcgugguccaccgacggucaaacguuguucgcuggauacuccgauaauacuauucgugucuggcaaguuucug

SEQ ID NO: 36 - RNA, 288 nt, Artificial Sequence
  dsRNA-SiGNBP-D
cagaaacuugccagacacgaauaguauuaucggaguauccagcgaacaacguuugaccgucgguggaccacgcgagggacaaacagagagguggcucugcuuuacuaguugcagacacaaccucagguuucaguucuucaaccauuuccuuagucucaagaucccauaucuuaauccaugguccgaaugcggcgcacagccaauaacgauuaggacuaaagcauaaugccguaaugaugucauuauggucuaaaguguggagauguuuuccaucauucagaucccaca

SEQ ID NO: 37 - RNA, 361 nt, Artificial Sequence
  dsRNA-SiGNBP-E
augaccgagacuuuacagcugagagggacgcuucgcggccacaauggaugggucacgcaaaucgcgacaaacccgaaauauccagacaugauuuugucuucuucacgugauaagacucugauuguguggaaauugacucgugaugaagcuaacuaugguaucccgcagaagcgucucuauggucacucacacuucauaagugauguaguucuuucaucugaugguaacuacgcucugucugguucaugggacaaaacauugcgucuuugggaucucgcagcaggucguacgaccagacgauuugaagaccauaccaaggauguuuugagugucgcuuuuuccguggacaaucgucagauug

SEQ ID NO: 38 - RNA, 361 nt, Artificial Sequence
  dsRNA-SiGNBP-E
caaucugacgauuguccacggaaaaagcgacacucaaaacauccuugguauggucuucaaaucgucuggucguacgaccugcugcgagaucccaaagacgcaauguuuugucccaugaaccagacagagcguaguuaccaucagaugaaagaacuacaucacuuaugaagugugagugaccauagagacgcuucugcgggauaccauaguuagcuucaucacgagucaauuuccacacaaucagagucuuaucacgugaagaagacaaaaucaugucuggauauuucggguuugucgcgauuugcgugacccauccauuguggccgcgaagcgucccucucagcuguaaagucucggucau

SEQ ID NO: 39 - RNA, 599 nt, Artificial Sequence
  dsRNA-SiGNBP-F
cguggacaaucgucagauuguuuccgguucgcgagacaagacaauuaaauuguggaauacauuggccgaaugcaaguauaccauccaggaugaugggcauacagauugggucagcugugugcgcuucucccccaaucaugcaaaucccaucauugucucugcaggcugggaucgcguugucaaggucuggaacuuaacgaacugcagguugaagaucaaucauaguggucauacaggauaucugaauacaguuacgguaucgccugauggaucacucugcgcauccgguggaaaagauugcaaagcuaugcugugggaucugaaugauggaaaacaucuccacacuuuagaccauaaugacaucauuacggcauuaugcuuuaguccuaaucguuauuggcugugcgccgcauucggaccauggauuaagauaugggaucuugagacuaaggaaaugguugaagaacugaaaccugagguugugucugcaacuaguaaagcagagccaccucucuguuugucccucgcgugguccaccgacggucaaacguuguucgcuggauacuccgauaauacuauucgugucuggcaaguuucug

SEQ ID NO: 40 - RNA, 599 nt, Artificial Sequence
  dsRNA-SiGNBP-F
cagaaacuugccagacacgaauaguauuaucggaguauccagcgaacaacguuugaccgucgguggaccacgcgagggacaaacagagagguggcucugcuuuacuaguugcagacacaaccucagguuucaguucuucaaccauuuccuuagucucaagaucccauaucuuaauccaugguccgaaugcggcgcacagccaauaacgauuaggacuaaagcauaaugccguaaugaugucauuauggucuaaaguguggagauguuuuccaucauucagaucccacagcauagcuuugcaaucuuuuccaccggaugcgcagagugauccaucaggcgauaccguaacuguauucagauauccuguaugaccacuaugauugaucuucaaccugcaguucguuaaguuccagaccuugacaacgcgaucccagccugcagagacaaugaugggauuugcaugauugggggagaagcgcacacagcugacccaaucuguaugcccaucauccuggaugguauacuugcauucggccaauguauuccacaauuuaauugucuugucucgcgaaccggaaacaaucugacgauuguccacg

SEQ ID NO: 41 - DNA, 20 bp, Artificial Sequence
  Primer
taatacgactcactataggg